(19) United States
(12) Patent Application Publication
(10) Pub. No.: US 2008/0132772 A1
(43) Pub. Date: Jun. 5, 2008

(54) Medical electrode

(76) Inventors: Burrhus Lang, Innsbruck (AT); Richard Webb, Minchinhampton (GB)

Correspondence Address: SEYFARTH SHAW LLP, WORLD TRADE CENTER EAST, TWO SEAPORT LANE, SUITE 300, BOSTON, MA, 02210-2028, US

(21) Appl. No.: 11/804,825

(22) Filed: May 21, 2007

Related U.S. Application Data
(63) Continuation of application No. PCT/AT2005/000468, filed on Nov. 22, 2005.

(30) Foreign Application Priority Data
Nov. 22, 2004 (AT) A 1957/2004

(51) Int. Cl. A61N 1/04

(52) U.S. Cl. 600/392

(57) ABSTRACT

This application comprises a plane medical electrode for dissipating signals from the human skin having a carrier element and at least one tapping pad projecting over the outer edge of the carrier element, wherein at least one contact point for picking up the signal from a patient's skin is provided in the area of said carrier element and a means for transferring/relaying the signal to a signal-acquisition or signal-processing unit is provided laterally offset from said contact point on said tapping pad, wherein the at least one contact point for picking up the signal from the skin is connected to the means for transferring/relaying the signal by a signal conductor, characterised in that to said contact point at least one additional means for transferring/relaying the signal to a signal-acquisition or signal-processing unit is assigned, said means being provided laterally offset on a tapping pad.

### RELATED APPLICATIONS

This application claims priority from PCT/AT2005/000468, filed on Nov. 22, 2005, which claims priority Austrian Application No. A 1957/2004, filed Nov. 22, 2004, is incorporated herein by reference

### BACKGROUND OF THE INVENTION

The invention concerns a medical electrode for dissipating signals from the human skin with at least one contact point for picking up the signal from a patient's skin and, laterally offset relative to the contact point, a means for transferring/relaying the signal to a signal-acquisition or signal-processing unit, the at least one contact point for collecting the signal from the skin being connected to the means for transferring or relaying the signal by a signal conductor.

Such electrodes have long been known, contact between the patient's skin and the signal conductor generally being made at the contact point with an electrically conductive gel. Laterally offsetting the means for transferring/relaying the signal enables gel to be prevented from getting on to the electrode's adhesive surface under the pressure exerted during connection of an electrode cable, as has often happened with electrodes where the means for transferring/relaying the signal is located centrally, i.e. at the contact point with the patient's skin.

### BRIEF SUMMARY OF THE INVENTION

The object of the invention is to create an improved, universally applicable medical electrode.

This is accomplished according to the invention by assigning to at least one contact point at least one additional laterally offset means for transferring/relaying the signal to a signal-acquisition or signal-processing unit. Advantageously, at least two of the means for transferring/relaying the signal to a signal-acquisition or signal-processing unit are of different type.

This enables the medical electrode according to the invention to be used irrespective of the type of terminals on the signal-acquisition or signal-processing unit. This is important because different organisations such as the rescue services, the Red Cross and hospitals use different signal-acquisition/processing units with differing terminals. It has therefore been necessary hitherto to produce several lots of the different types of medical electrode, each fitted with the terminal connection appropriate for one signal-acquisition/processing unit. Fitting medical electrodes with two laterally offset, dissimilar means for transferring/relaying the signal according to the invention avoids this disadvantage and yields a low-cost, universally applicable medical electrode.

In a first embodiment of the invention the at least two means for transferring/relaying the signal assigned to the one contact point are connected to the contact point by a common signal conductor, while in an alternative embodiment of the invention the at least two means for transferring/relaying the signal assigned to the single contact point are connected to the contact point by separate signal conductors, said separate signal conductors being galvanically interconnected, preferably at the one contact point.

What therefore matters first and foremost is not how the laterally offset means for transferring/relaying the signal are connected to the one contact point for collecting the signal from the skin. Fundamental to the invention is rather the fact that two laterally offset means for transferring/relaying the signal are assigned to the one contact point.

In a preferred embodiment of the invention the medical electrode may be provided with a carrier element and at least one tapping pad which projects beyond the outer edge of the carrier element, at least one means for transferring/relaying the signal to a signal-acquisition or signal-processing unit being located on this tapping pad. In other words, the medical electrode according to the invention thus has two non-central means for transferring/relaying the signal, at least one of the said means being located on a preferably flexible strip, so ensuring a stable and interference-free signal. If the signal is relayed via an electrode cable, the construction of the medical electrodes according to the invention ensures that even movements of this electrode cable do not impair accurate and reliable recording of the signal.

Another embodiment of the invention provides for the medical electrode to have at least one tapping pad on which at least two means for transferring/relaying the signal to a signal-acquisition or signal-processing unit are provided. It is also possible for the medical electrode to have several tapping pads, on each of which at least one means for transferring/relaying the signal to a signal-acquisition or signal-processing unit is provided; it has been found especially advantageous for the medical electrode to have several tapping pads, on each of which precisely one means for transferring/relaying the signal to a signal-acquisition or signal-processing unit is provided.

Disposing several non-central means for transferring/relaying the signal on a tapping pad, or one non-central means on a tapping pad each, ensures that, when the signal is transferred/relayed via the non-central terminals, direct pressure is not exerted on the patient's body, especially when the body of the medical electrode is free from laterally offset means for transferring/relaying the signal, i.e. all the medical electrode's non-central terminals are arranged on tapping pads.

Another embodiment of the invention provides at the contact point for collecting the signals from the skin an additional means for transferring/relaying the signal to a signal-acquisition or signal-processing unit, whereby the number of preferably dissimilar means for transferring/relaying the signal can be increased.

In a preferred embodiment of the invention the means for transferring/relaying the signal to a signal-acquisition or signal-processing unit include a pushbutton and/or plug, preferably a banana plug, and/or are constructed so that a clip, preferably a crocodile clip, can be attached to them, and/or for non-contact transfer of the signal. By suitably combining these different means an effectively universally applicable medical electrode is obtained, in which the number of non-central terminals will be limited in practice to two or three so as to keep the size of the medical electrode suitably small.

In a preferred embodiment of the invention the novel medical electrode possesses a flexible insulating carrier element with an opening to accommodate an electrically conductive gel, there being on one side of the carrier element, preferably covering the whole surface, an adhesive layer for attaching the electrode to the patient's skin and, on the side of the carrier element opposite to the adhesive, a signal conductor, preferably in the form of a signal-conducting layer, which can be brought into contact with the patient's skin at the contact point via the electrically conductive gel. A covering layer, preferably in the form of a label, is provided over the signal conductor and carrier element, said covering layer having a tapping pad projecting beyond the outer edge of the carrier element, on which tapping pad two dissimilar means for transferring/relaying the signal to a signal-acquisition or signal-processing unit are arranged.

### BRIEF DESCRIPTION OF THE DRAWINGS

Further details of the invention and the advantages obtained by it will be apparent from the explanation below of the embodiments of a medical electrode according to the invention illustrated in the drawing. Of the figures comprising the drawing

FIG. 1 shows schematically a cross section through a first embodiment of a medical electrode according to the invention;

FIG. 2 shows schematically an exploded view of a medical electrode according to the invention; and

FIG. 3a-3d show further embodiments of a medical electrode according to the invention.

### DETAILED DESCRIPTION

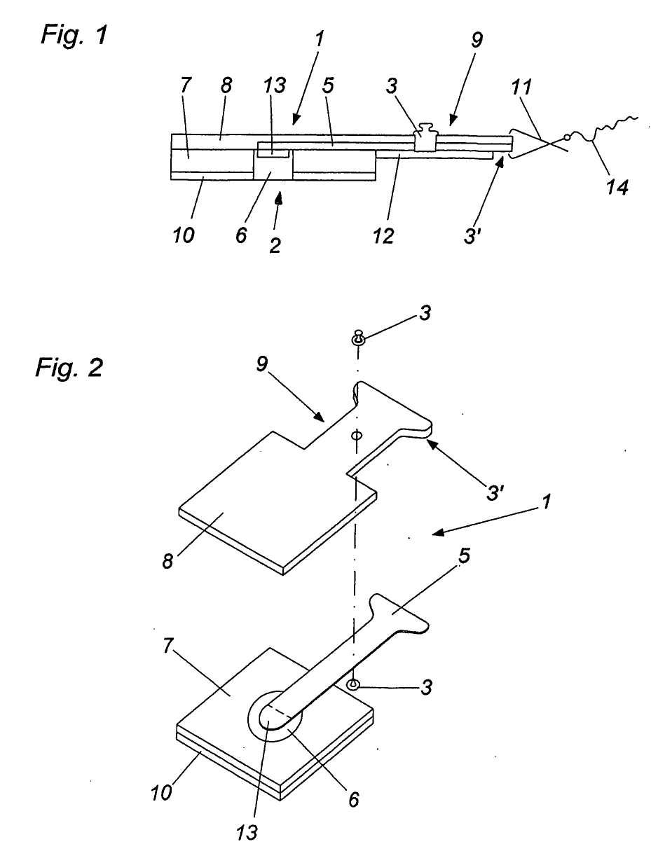

The medical electrode 1 illustrated in FIGS. 1 and 2 has—from top to bottom—an adhesive layer 10 made of a skin-compatible adhesive, a flexible insulating carrier element 7 of polymer foam, a metallic signal conductor 5, a carbon conductor for example, and a label-form covering layer 8. The signal conductor 5 and a section of the covering layer 8 which covers the signal conductor 5 project laterally relative to the carrier element 7, so forming a tapping pad 9.

On this tapping pad 9 two means 3, 3′ for transferring/relaying a signal collected from a patient's skin at the contact point 2 are provided laterally offset relative to the contact point 2. One means 3 takes the form of a pushbutton/plug and the other 3′ is designed to receive a crocodile clip 11 attached to an electrode cable 14. On the side of the tapping pad 9 facing the carrier element 7 there is at least in parts a cover 12, intended on the one hand to protect the signal conductor 5 from interference and on the other to prevent the signal conductor 5 from contacting the patient's skin.

Signal collection from the patient's skin takes place at the contact point 2 via an electrically conductive gel 6 accommodated in an opening in the flexible carrier element 7 and in contact with the signal conductor 5. For improved conductivity between the electrically conductive gel 6 and the signal conductor 5 the latter may also be given a silver/silver chloride coating in the area of the electrically conductive gel 6 on the side facing the electrically conductive gel.

Even if the signal conductor 5 in the embodiment shown in FIG. 1 and 2 is purely metallic, it goes without saying that the signal conductor 5 could also be printed-on at the underside of the covering layer 8 for example. Another possibility would be to insulate the signal conductor 5 on its side facing the carrier element 7, for example by making the signal conductor 5 as a two-layer membrane, one side of the membrane being electrically conductive and the other electrically isolating. In this case the tapping pad 9 could be formed straight from the projecting part of the signal conductor 5 made as a two-layer membrane, so that the covering layer 8 would only need to be the size of the carrier element 7.

The fact that the two dissimilar means 3, 3′ for transferring/relaying the signal are disposed non-centrally on the electrode 1 yields an improved, i.e. more stable, ECG signal. Not only is attachment of the means 3, 3′ to the terminals of the signal-acquisition or signal-processing unit simple, the non-central arrangement of the means 3, 3′ also makes the medical electrode 1 according to the invention more tolerant of movements in the electrode cables 14. Moreover, the two non-central means 3, 3′ for transferring/relaying the signals make parallel dissipation of signals possible.

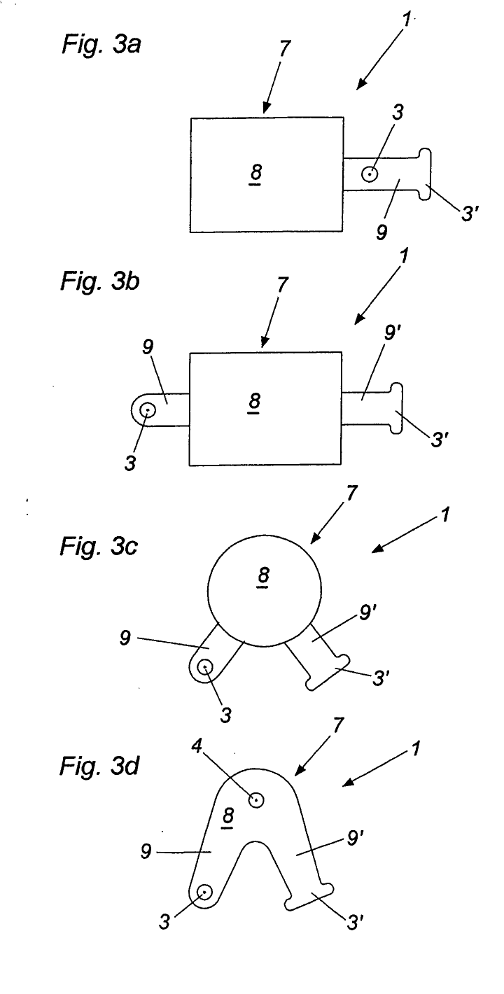

FIG. 3a to 3d show by way of example other possible implementations of a medical electrode 1 according to the invention. The electrode 1 depicted in FIG. 3a has a tapping pad 9 which projects relative to the carrier element 7, on which tapping pad 9 two dissimilar means 3, 3′ are arranged for transferring/relaying the signal.

FIGS. 3b and 3c show an electrode 1 with a rectangular (FIG. 3b) and round (FIG. 3c) carrier element 7, on each of which are arranged two tapping pads 9, 9′, each tapping pad 9, 9′ having precisely one pick-up element 3, 3′ for transferring/relaying the signals. In both of these specimen embodiments it would of course also be possible to provide more than one means 3, 3′ for transferring/relaying the signal on one or both tapping pads 9, 9′.

FIG. 3d shows a V-shaped electrode 1, one non-central means 3, 3′ for transferring/relaying the signal being provided at the end of each arm of the V-shaped electrode 1. This embodiment of the invention additionally has one central means 4 for transferring/relaying the signal.

The illustrated embodiments of medical electrodes are of course not to be understood restrictively, but are instead just a few examples taken from numerous possible ways of implementing the inventive concept of a medical electrode with at least two non-central means for transferring/relaying signals collected from a patient's skin.

What is claimed is:

1. A plane medical electrode for dissipating signals from the human skin having a carrier element and at least one tapping pad projecting over the outer edge of the carrier element, wherein at least one contact point for picking up the signal from a patient's skin is provided in the area of said carrier element and a means for transferring/relaying the signal to a signal-acquisition or signal-processing unit is provided laterally offset from said contact point on said tapping pad, wherein the at least one contact point for picking up the signal from the skin is connected to the means for transferring/relaying the signal by a signal conductor, characterised in that to said contact point at least one additional means for transferring/relaying the signal to a signal-acquisition or signal-processing unit is assigned, said means being provided laterally offset on a tapping pad.

2. Medical electrode according to claim 1, characterised in that the at least two means assigned to the one contact point are connected to the contact point by a common signal conductor.

3. Medical electrode according to claim 1, characterised in that the at least two means assigned to the one contact point are connected to the one contact point by separate signal conductors, the separate signal conductors being galvanically interconnected.

4. Medical electrode according to claim 3, characterised in that the separate signal conductors are galvanically interconnected at the one contact point.

5. Medical electrode according to claim 1, characterised in that the means for transferring/relaying the signal are formed as a pushbutton, a plug, for connection of a clip or for a contact-less transfer of the signal.

6. Medical electrode according to claim 5, characterised in that said plug is a banana plug.

7. Medical electrode according to claim 5, characterised in that the means are formed for the connection of a crocodile clip.

8. Medical electrode according to claim 1, characterised in that the at least two means for transferring/relaying the signal are provided on one tapping pad.

9. Medical electrode according to claim 8, characterised in that a first means comprises a pushbutton or a plug and a second means is formed for connection of a clip, wherein concerning the carrier element the second means is positioned abroad on the tapping pad.

10. Medical electrode according to claim 1, characterised in that the medical electrode has several tapping pads, at least one means for transferring/relaying the signal to a signal-acquisition or signal-processing unit is provided on each tapping pad.

11. Medical electrode according to claim 10, characterised in that precisely one means for transferring/relaying the signal to a signal-acquisition or signal-processing unit is provided on each tapping pad.

12. Medical electrode according to claim 1, characterised in that an additional means is provided for transferring/relaying the signal to a signal-acquisition or signal-processing unit, said means being provided at the contact point for collecting the signal from the skin.

13. Medical electrode according to claim 1, characterised in that at least two of the means for transferring/relaying the signal to a signal-acquisition or signal-processing unit are of different type.

14. Medical electrode according to claim 1, characterised by a flexible insulating carrier element with an opening to accommodate an electrically conductive gel, an adhesive layer being provided on one side of the carrier element for attaching the electrode to the patient's skin, and a signal conductor being provided on the side of the carrier element opposite the adhesive layer, said signal conductor could be brought into contact with the patient's skin at the contact point via the electrically conductive gel, as well as a covering layer being arranged over the signal conductor and carrier element, said covering layer has a tapping pad projecting over the outer edge of the carrier element on which tapping pad two dissimilar means are provided for transferring/relaying the signal to a signal-acquisition or signal-processing unit.

15. Medical electrode according to claim 12, characterised in that the covering layer is in the form of a label.

16. Medical electrode according to claim 12, characterised in that the adhesive layer is provided over the full area of the carrier element.

17. Medical electrode according to claim 12, characterised in that the signal conductor is in the form of a signal-conducting layer.

18. A system for dissipating signals from the human skin comprising a plane medical electrode and a signal acquisition or signal processing unit, the medical electrode having a carrier element and at least one tapping pad, which projects over the outer edge of said carrier element and comprises a means for transferring/relaying the signal picked up from the human skin at a contact point provided in the area of the carrier element, characterised in that two means for transferring/relaying the signal are provided on said tapping pad, wherein a first means comprises a pushbutton or a plug and a second means is formed for connection of a clip.

19. System according to claim 18, characterised in that said second means is formed for the connection of a crocodile clip.

20. System according to claim 18, characterised in that concerning the carrier element the second means is positioned abroad on the tapping pad.
